(19) United States
(12) Patent Application Publication
(10) Pub. No.: US 2016/0090397 A1
(43) Pub. Date: Mar. 31, 2016

(54) Se-Adenosyl-L-Selenohomocysteine Selenoxide As A Modulator Of Methyltransferase And Other Activities

(71) Applicant: Northeastern University, Boston, MA (US)

(72) Inventors: Zhaohui Sunny Zhou, Wellesley, MA (US); Richard I. Duclos, JR., Quincy, MA (US); Dillon C. Cleary, Middleboro, MA (US); Kalli C. Catcott, Boston, MA (US)

(21) Appl. No.: 14/871,099

(22) Filed: Sep. 30, 2015

Related U.S. Application Data
(60) Provisional application No. 62/057,956, filed on Sep. 30, 2014.

(51) Int. Cl. C07H 19/16; C12Q 1/48

(52) U.S. Cl. CPC C07H 19/16; C12Q 1/48; G01N 2333/91017; G01N 2500/20; G01N 2500/04

(57) ABSTRACT

The invention relates to a compound of the structural formula

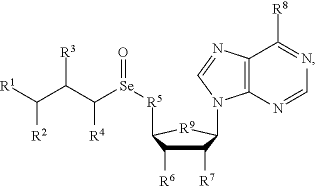

or a hydrate or an isotope thereof. The invention also relates to a preparation method thereof and methods of providing dietary organoselenium, inactivating an enzyme, modulating the activity of a protein (e.g., methyltransferase) or nucleic acid, identifying a methyltransferase that reacts with compound, and oxidizing a methyltransferase-reactive substrate.

### RELATED APPLICATION

This application claims the benefit of U.S. Provisional Application No. 62/057,956, filed on Sep. 30, 2014. The entire teachings of the above application are incorporated herein by reference.

### GOVERNMENT SUPPORT

This invention was made with government support under Grant Number 1R01GM101396 awarded by the National Institute of Health (NIH). The government has certain rights in the invention.

### BACKGROUND OF THE INVENTION

Selenium is an essential micronutrient for all animals and many other living organisms. However, a high level of selenium is toxic. Thus, selenium metabolites should be maintained within a fairly narrow concentration range of adequacy for the biosynthesis of the over 25 human selenoproteins to balance deficiency and toxicity.

Organoselenium metabolites are only present in trace amounts in living organisms, relative to the well-known sulfur analogs that include the amino acids L-methionine and L-cysteine, the biological methyl donor S-adenosyl-L-methionine (AdoMet or SAM), and the byproduct of methylation S-adenosyl-L-homocysteine (AdoHcy or SAH). Oxidations of S-adenosylhomocysteine (SAH) are reported to give the sulfoxide (SAHO) and the corresponding sulfone. The sulfoxide and sulfone have not been detected as metabolites in vivo, but as close structural analogs of AdoMet, these analogs are methyltransferase enzyme inhibitors in vitro. As examples, SAHO is an inhibitor of catechol-O-methyltransferase (COMT), phenylethanolamine N-methyltransferase (PNMT), histamine N-methyltransferase (HMT), protein methyltransferase II, viral mRNA methyltransferases, and E. coli cyclopropane fatty acid synthase.

The redox biochemistry of methionine is well understood. The selenium analog of methionine (selenomethionine) is also easily oxidized with biological oxidants such as hydrogen peroxide to give a mixture of selenoxide (selenomethionine selenoxide) and hydrate in the neutral pH range. NMR data are pH dependent, and only single compounds are seen at low pH and at high pH. These data are consistent with studies of other selenoxides. Selenomethionine selenoxide is homogeneous by HPLC and stable at ambient temperature. Homocysteine, selenohomocysteine, cysteine, and selenocysteine also undergo oxidations.

While Se-adenosylselenohomocysteine (SeAH) and S-adenosylhomocysteine (SAH) have been previously characterized, little is known about the redox chemistry of SeAH and the biochemical activity of the corresponding selenoxide.

Thus, not only is further investigation into the biochemical activity and redox chemistry of SeAH needed, but new and different organoselenium metabolites for addressing selenium deficiency are also desirable.

### SUMMARY OF THE INVENTION

One embodiment of the invention relates to an organoselenium compound having a structural formula

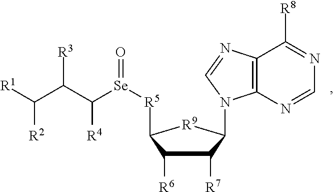

or a hydrate or an isotope thereof, wherein R¹ is COOH, NH₂, CH₂OH, CHO, CONH₂, Cl, Br, I, H, O, OH, N₃, CH₃ or CN; R² is NH₂, COOH, CH₂OH, CHO, CONH₂, Cl, Br, I, H, O, OH, N₃, CH₃ or CN; R³ is H or a linear or branched C₁-C₄ alkyl; R⁴ is H or a linear or branched C₁-C₄ alkyl; R⁵ is CH₂, —CH(CH₃), C(CH₃)₂ or C₂H₄; R⁶ is OH, O or a linear or branched C₁-C₄ alkoxy; R⁷ is OH, O or a linear or branched C₁-C₄ alkoxy; R⁸ is NH₂, COOH, Cl, Br, I, H, O, OH, N₃, CH₃ or CN; and R⁹ is O, N, S or CH₂. The present inventors discovered this organoselenium compound upon their investigation into the biochemical activity and redox chemistry of SeAH.

Another embodiment of the invention relates to a method of preparing the organoselenium compound of the invention, wherein a precursor compound having a structural formula

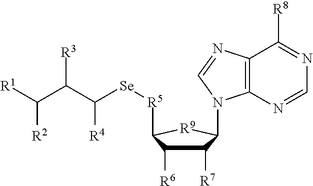

is oxidized.

An additional embodiment of the invention relates to a method of providing dietary organoselenium to a subject in need thereof, wherein a composition containing the organoselenium compound of the invention is administered to the subject.

Another embodiment of the invention relates to a method of inactivating an enzyme, wherein the enzyme is contacted, under physiological conditions, with the organoselenium compound of the invention, and wherein the enzyme has at least one of an accessible cysteine or oxidizable functional group in an active site of the enzyme.

An additional embodiment of the invention relates to a method of modulating the activity of a methyltransferase, wherein the methyltransferase is contacted, under physiological conditions, with the organoselenium compound of the invention.

Another embodiment of the invention relates to a method of identifying a methyltransferase that reacts with the organoselenium compound of the invention, wherein a methyltransferase is contacted, under physiological conditions, with the organoselenium compound of the invention, thereby oxidizing the methyltransferase and producing a 16 Da mass shift in the methyltransferase, and wherein the methyltransferase is identified by the 16 Da mass shift.

An additional embodiment of the invention relates to a method of oxidizing a methyltransferase-reactive substrate, wherein the substrate is contacted, under physiological conditions, with a methyltransferase and the organoselenium compound of the invention.

Attributes of the invention include, but are not limited to, using the organoselenium compound of the invention as a dietary micronutrient selenium supplement, using the organoselenium compound of the invention as an inhibitor of enzymes that utilize SAH and SAM/AdoMet, using the organoselenium compound of the invention as a selenium analog of SAH having a different HPLC retention to probe SAH metabolic studies in vitro, using the organoselenium compound of the invention as a methyltransferase modifier, and using the organoselenium compound of the invention as an alternative methyltransferase substrate.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 depicts an absorbance spectrum for Se—SAHO as a thiopurine methyltransferase (TPMT) substrate wherein the oxidation of thionitrobenzoate (TNB) by H₂O₂ causes a clear shift in absorbance maxima from ˜410 nm to ˜325 nm.

FIGS. 2A-2B depict an absorbance spectrum (2A) and a plot over time (2B) for Se—SAHO as a TPMT substrate wherein Se-SAHO is able to oxidize TNB causing a shift in absorbance maxima.

FIG. 3 depicts a scheme for Se-SAHO as a TPMT substrate (right) and corresponding spectra indicating Se-SAHO and Se-SAH peaks (left).

FIG. 4 depicts a scheme for Se-SAHO as a catechol-O-methyltransferase (COMT) substrate.

FIG. 5 depicts corresponding spectra for Se-SAHO as a COMT substrate that indicate epinephrine and epinephrine oxidation product adrenochrome.

FIG. 6 depicts spectra (top) and an absorbance plot (bottom) for Se-AHO as a COMT substrate wherein the spectra compare the presence of the COMT substrate (middle spectrum) and absence of the COMT substrate (bottom spectrum).

FIG. 7 illustrates that SeAHO reacts with epinephrine to form adrenochrome at a rate dependent on COMT concentration wherein adrenochrome concentration versus time at various COMT concentrations is shown (top) and rates of adrenochrome formation versus COMT concentration is shown (bottom).

FIG. 8 depicts an HPLC analysis of the reduction of SeAHO to SeAH by glutathione.

FIG. 9 depicts the reduction of selenoxide by glutathione with corresponding spectra that indicate Se-AHO and Se-AH peaks.

### DETAILED DESCRIPTION OF THE INVENTION

A description of example embodiments of the invention follows.

A first embodiment of the invention relates to an organoselenium compound having a

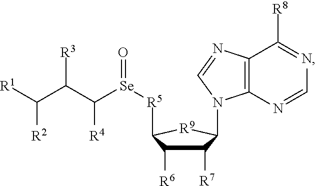

structural formula or a hydrate or an isotope thereof, wherein R¹ is COOH, NH₂, CH₂OH, CHO, CONH₂, Cl, Br, I, H, O, OH, N₃, CH₃ or CN; R² is NH₂, COOH, CH₂OH, CHO, CONH₂, Cl, Br, I, H, O, OH, N₃, CH₃ or CN; R³ is H or a linear or branched C₁-C₄ alkyl; R⁴ is H or a linear or branched C₁-C₄ alkyl; R^{5 is CH}₂, —CH(CH₃), C(CH₃)₂ or C₂H₄; R⁶ is OH, O or a linear or branched C₁-C₄ alkoxy; R⁷ is OH, O or a linear or branched C₁-C₄ alkoxy; R⁸ is NH₂, COOH, Cl, Br, I, H, O, OH, N₃, CH₃ or CN; and R⁹ is O, N, S or CH₂.

In one aspect of the first embodiment, R¹ is COOH, R² is NH₂, R³ is H, R⁴ is H and R⁵ is CH₂. In another aspect of the first embodiment, R⁵ is CH₂, R⁶ is OH, R⁷ is OH, R⁸ is NH₂ and ^{R}9 is O. In an additional aspect of the first embodiment, R³ is H, R⁴ is H, R⁵ is CH₂, R⁶ is OH, R⁷ is OH and R⁹ is O.

In three particular aspects of the first embodiment, the organoselenium compound can

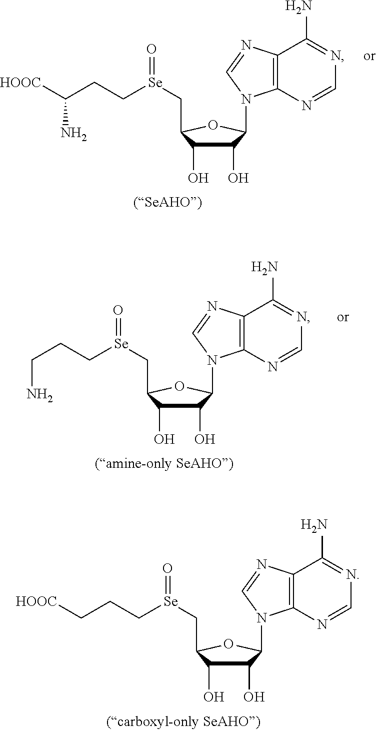

have the formula

The hydrate form of the organoselenium compound of the first embodiment can have the formula

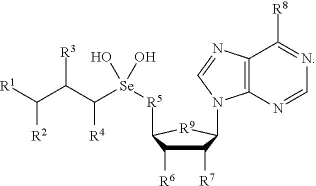

In yet another aspect of the first embodiment, the organoselenium compound can be represented by one or more of the following:

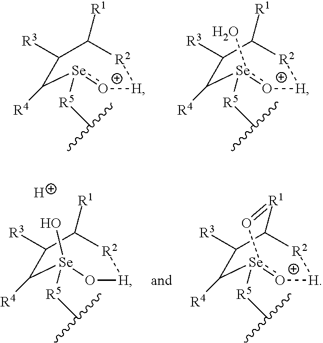

An additional aspect of the first embodiment relates to modifying the selenium atom of the organoselenium compound with various isotopes to exploit radio-tracing, nuclear magnetic resonance, or mass-shift analysis. Possible isotopes of selenium include ⁷⁴Se, ⁷⁵Se, ⁷⁶Se, ⁷⁷Se, ⁷⁸Se, ⁸⁰Se and ⁸²Se. Also, any of the nitrogen, carbon and hydrogen atoms of the organoselenium compound can be isotopic.

A second embodiment of the invention relates to a method of preparing the organoselenium compound of the invention, wherein a precursor compound having a structural formula

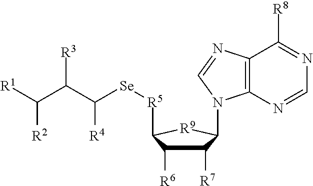

is oxidized, thereby obtaining the organoselenium compound of the invention. The oxidation can be performed via any known oxidation method, including but not limited to oxidation via the use of hydrogen peroxide, meta-chloroperoxybenzoic acid, and ozone.

In an aspect of the second embodiment R¹ is COOH, R² is NH₂, R³ is H, R⁴ is H, R⁵ is CH₂, R⁶ is OH, R⁷ is OH, R⁸ is NH₂ and R⁹ is O, and the preparation scheme is as follows

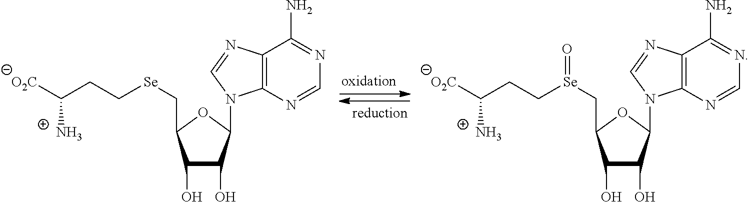

In another aspect of the second embodiment, R¹ is COOH, R² is NH₂, R³ is H, R⁴ is H, R⁵ is CH₂, R⁶ is OH, R⁷ is OH, R⁸ is NH₂ and R⁹ is O, and the preparation scheme includes any one or more of the synthesis routes depicted in the following scheme

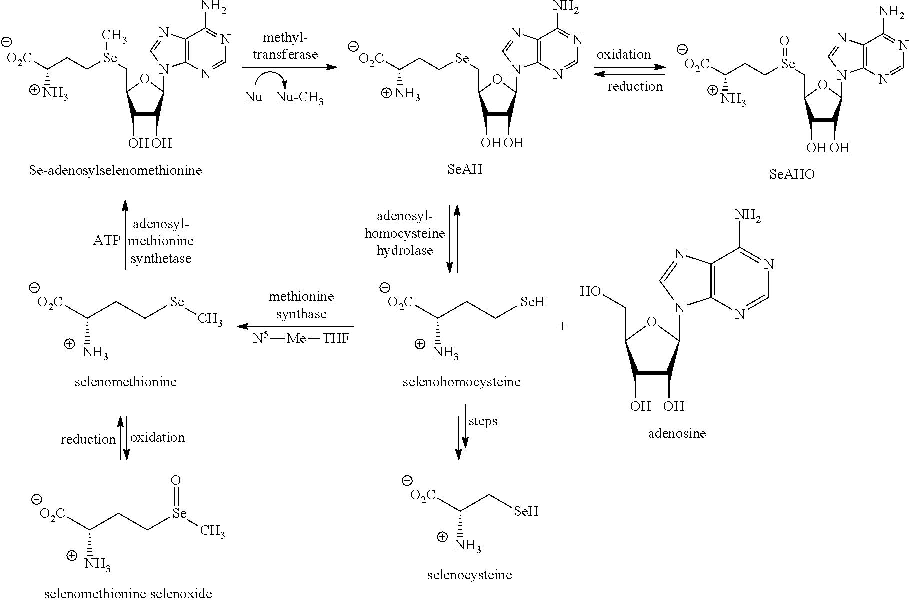

In yet another aspect of the second embodiment, R¹ is COOH, R² is NH₂, R³ is H, R⁴ is H, R⁵ is CH₂, R⁶ is OH, R⁷ is OH, R⁸ is NH₂ and R⁹ is O, and the preparation scheme includes the following scheme

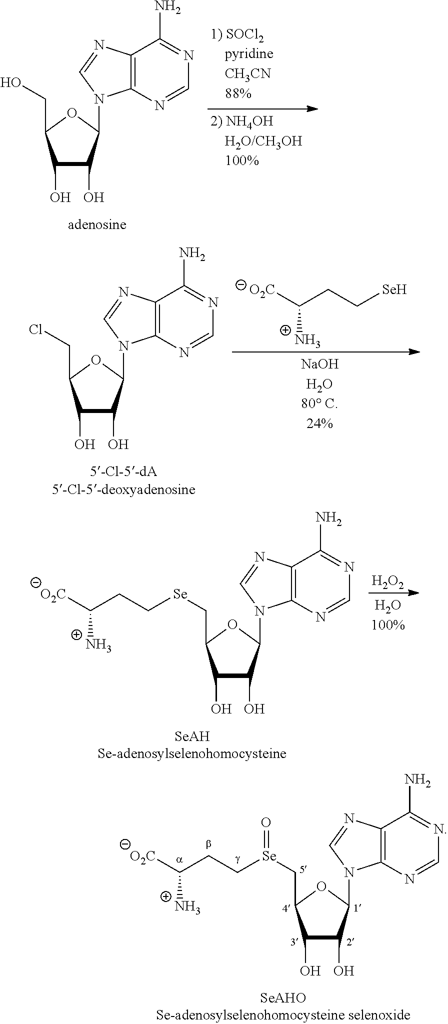

This preparation scheme can be performed without extraction and without column chromatography.

A third embodiment of the invention relates to a method of providing dietary organoselenium to a subject in need thereof, wherein the method comprises administering to the subject a composition comprising the organoselenium compound of the invention.

As used herein, the term “subject” encompasses mammals such as humans, non-human primates, livestock, companion animals (e.g., dogs and cats), and laboratory animals (e.g., rodents and lagamorphs). In a particular aspect, the subject is a human.

In an aspect of the third embodiment, the subject has a selenium imbalance. A selenium imbalance can be a deficiency of selenium or an excess of selenium. A selenium imbalance is one known cause of depression. Accordingly, in one embodiment, the subject has depression.

A skilled medical professional can determine whether a subject has a selenium imbalance and can determine an appropriate dosage and regimen for administering the organoselenium compound of the invention, which will vary according to characteristics of the subject, such weight, age, etc.

The composition comprising the organoselenium compound of the invention can be in any dosage form suitable for administration to a subject (e.g., oral (including buccal and sublingual), rectal, nasal, topical, pulmonary, vaginal, parenteral (including intramuscular, intraarterial, intrathecal, subcutaneous and intravenous), inhalation or insufflation).

For oral administration, the formulation will generally take the form of a tablet, capsule, or softgel capsule, or may be an aqueous or nonaqueous liquid solution, suspension (e.g., microsphere suspension), or syrup. Tablets and capsules are preferred oral administration forms. Tablets and capsules for oral use will generally include one or more commonly used carriers such as lactose and corn starch. Lubricating agents, such as magnesium stearate, are also typically added. When liquid suspensions (e.g., microsphere suspensions) are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents, and the like. In other aspects, the formulation can take the form of a powder that can be dissolved into an aqueous solution for administration. The aqueous or nonaqueous liquid solution can also be added to an aqueous solution for administration (e.g., liquid solution can be mixed with baby formula).

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can be prepared, for example, by dissolving, dispersing, etc., an active compound or conjugate as described herein and optional pharmaceutical adjuvants in an excipient, such as, for example, water, saline, aqueous dextrose, glycerol, ethanol, and the like, to thereby form a solution or suspension. If desired, the composition to be administered may also contain minor amounts of nontoxic auxiliary substances such as wetting or emulsifying agents, pH buffering agents, tonicifying agents, and the like, for example, sodium acetate, sorbitan monolaurate, triethanolamine sodium acetate, triethanolamine oleate, etc. Actual methods of preparing such dosage forms are known, or will be apparent, to those skilled in the relevant art.

A fourth embodiment of the invention relates to a method of inactivating an enzyme, wherein the method comprises contacting, under physiological conditions, the enzyme with the organoselenium compound of the invention, and wherein the enzyme has at least one of an accessible cysteine or oxidizable functional group in an active site of the enzyme.

In one aspect of the fourth embodiment, enzymes having an accessible cysteine group in an active site of the enzyme are adenosyl homocysteine hydrolase (EC 3.3.1.1), MGMT (EC 2.1.1.63), Dam (EC 2.1.1.72), and DNA(cytosine-5)-methyltransferase 1 (EC 2.1.1.37). Radical SAM enzymes have a conserved cysteine motif

In another aspect of the fourth embodiment, the enzyme having an oxidizable functional group in an active site of the enzyme is DNA(cytosine-5)-methyltransferase 1 (EC 2.1.1.37). Radical SAM enzymes have iron sulfur clusters that can be oxidized. Additionally, PNMT (EC 2.1.1.28) and HMT (EC 2.1.1.43) have active site tyrosine residues.

In yet another aspect of the fourth embodiment, the enzyme is an enzyme that binds S-adenosylmethionine, for example a methyltransferase (with enzyme commission number, 2.1.1.xxx), for instance catechol-O-methyltransferase (COMT, EC 2.1.1.6) or thiopurine methyltransferase (TPMT, EC 2.1.1.67). Additional SAM binding enzymes include adenosylmethionine decarboxylase (EC 4.1.1.50) and radical SAM enzymes (pFAM: PF04055).

A fifth embodiment of the invention relates to a method of modulating (e.g., increasing or decreasing) the activity of a methyltransferase, wherein the method comprises contacting, under physiological conditions, a methyltransferase with the organoselenium compound of the invention.

As used herein, the term “methyltransferase” refers to transferase class enzymes that are able to transfer a methyl group from a donor molecule to an acceptor molecule, e.g., an amino acid residue of a protein or a nucleic base of a DNA molecule. Methyltransferases typically use a reactive methyl group bound to sulfur in S-adenosyl methionine (SAM) as the methyl donor. In some embodiments, a methyltransferase described herein is a protein methyltransferase. In some embodiments, a methyltransferase described herein is a histone methyltransferase. Histone methyltransferases (HMT) are histone-modifying enzymes, (including histone-lysine N-methyltransferase and histone-arginine N-methyltransferase), that catalyze the transfer of one or more methyl groups to lysine and arginine residues of histone proteins. In certain embodiments, a methyltransferase described herein is a histone-arginine N-methyltransferase.

In an aspect of the fifth embodiment, the methyltransferase is a S-adenosylmethionine dependent methyltransferase.

In another aspect of the fifth embodiment, the methyltransferase is catechol-O-methyltransferase (COMT) or thiopurine methyltransferase (TPMT).

In yet another aspect of the fifth embodiment, the organoselenium compound of the invention inhibits the activity of the methyltransferase.

In a further aspect of the fifth embodiment, the organoselenium compound of the invention activates the activity of the methyltransferase.

A sixth embodiment of the invention relates to a method of identifying a methyltransferase that reacts with the organoselenium compound of the invention, wherein the method comprises contacting, under physiological conditions, a methyltransferase with the organoselenium compound of the invention, thereby oxidizing the methyltransferase and producing a 16 Da mass shift in the methyltransferase, and then identifying the methyltransferase by the 16 Da mass shift. Mass spectrometry could be used to detect the 16 Da mass shift.

A seventh embodiment of the invention relates to a method of oxidizing a methyltransferase-reactive substrate, wherein the method comprises contacting, under physiological conditions, the substrate with a methyltransferase and the organoselenium compound of the invention. Examples of suitable methyltransferase-reactive substrates include, but are not limited to, small molecules (such as catechols, purines, mecaptans, and captopril and its derivatives), xenobiotics, drugs and drug metabolites, lipids, carbohydrate, peptides, proteins (such as histones, MAP3k2 (EC 2.7.11.25), and p53 (UniProt: PO4637)), nucleic acids (such as the C5 position of cytosine, and the N6 position of adenine), and other methyltransferase targets.

### EXEMPLIFICATION

### Example 1

SeAHO was prepared in 21% overall yield according to:

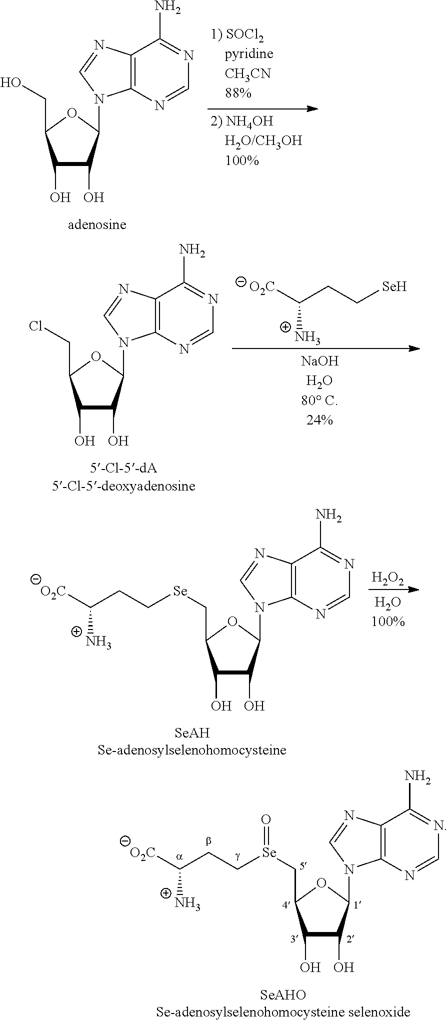

(Duclos Jr, R. I.; Cleary, D. C.; Catcott, K. C.; Zhou, Z. S. Journal of Sulfur Chemistry 2015, 36, 135-144; herein incorporated by reference).

Adenosine was first converted to 5′-chloro-5′-deoxyadenosine (5′-Cl-5′-dA) by the reported method (Scovill, J. P.; Thigpen II, D. L.; Lemley, P. V. Phosphorus, Sulfur, and Silicon 1993, 85, 149-52) in 88% yield. Reaction of 5′-Cl-5′-dA with L-selenohomocysteine, prepared by our previously reported method (Zhou, Z. S.; Smith, A. E.; Matthews, R. G. Bioorg. Med. Chem. Lett. 2000, 10, 2471-5; Willnow, S.; Martin, M.; Luscher, B.; Weinhold, E. ChemBioChem 2012, 13, 1167-73), gave SeAH that was isolated in 24% yield after two recrystallizations. Partial protonation or a conformational difference in the adenosine moiety may account for the two sets of aromatic and glycosidic protons seen in pH 3 D₂O phosphate buffer that was not seen in deuterated acetic acid solution by proton NMR. The oxidations of both this selenium analog SeAH and the commercially available sulfur analog SAH were studied under the same conditions.

For the sulfur analog, hydrogen peroxide oxidation of SAH in acetic acid solution according to a literature report (Guerard, C.; Breard, M.; Courtois, F.; Drujon, T.; Ploux, O. Bioorg. Med. Chem. Lett. 2004, 14, 1661-4) gave nearly equal ratios of the R and S isomers at the newly chiral sulfur center for the product SAHO. These two diastereomers had distinctly different resonances in the proton NMR for the diastereotopic α, γ, 2′, 3′, 4′, 5′, and for one aromatic resonance (see Table 1). The diastereomeric (at sulfur) mix of sulfoxides SAHO had 975 and 998 cm⁻¹ bands in the IR that are characteristic of sulfoxide stretching. For example, our IR of dimethylsulfoxide (DMSO, data not shown) showed strong absorptions at 1018 and 1042 cm⁻¹.

The corresponding hydrogen peroxide oxidation of the selenium analog SeAH to SeAHO was first performed in acetic acid solution. Vacuum transfer of an acetic acid solution gave a solid sample of SeAHO that was characterized by melting point and IR. Some carbonyl stretch was observed at 1777 cm⁻¹ in the IR spectrum, characteristic of α-amino carboxylic acids at low pH. The selenoxide SeAHO also showed characteristic Se═O stretching bands in the IR at 847 and 878 cm⁻¹. The ¹H NMR of selenoxide SeAHO in acetic acid-d₄/H₂O/H₂O₂ 600:9:1, an organic acidic environment near the pKa's of the carboxylic acid group and the adenine residue, was clean and assignable for the selenoxide SeAHO. The selenoxide SeAHO has a distinctly different conformation than the corresponding sulfoxide SAHO as evidenced by the dramatic downfield shift of the α-proton in the NMR (see Table 1). The hydrolysis of the Cl′-adenine bond of SeAHO in the acetic acid/water was followed over several hours by ¹H NMR (data not shown).

TABLE 1
¹H Chemical shifts of SAH, SeAH, and SAHO in CD₃CO₂D;
and, of SeAHO in 97:3 CD₃CO₂D/30% aqueous H₂O₂.

| | | SAHO | | |
|---|---|---|---|---|
| Proton | SAH | 3 (X = S) | SeAH | SeAHO |
| assign. | 2 (X = S) | (1:1 mix) | 2 (X = Se) | 3 (X = Se) |
| α | 4.18 | 4.15, 4.17 | 4.16 | 4.58-4.67 |
| βa | 2.27-2.34 | 2.49-2.58 | 2.32-2.39 | 2.74-2.83 |
| βb | 2.16-2.23 | 2.44-2.53 | 2.22-2.30 | 2.60-2.69 |
| γa | 2.76-2.85 | 3.28-3.37, 3.28-3.37 | 2.74-2.84 | 3.84-3.95 |
| γb | 2.76-2.85 | 3.19-3.25, 3.28-3.37 | 2.74-2.84 | 3.84-3.95 |
| 1′ | 6.16 | 6.19 | 6.16 | 6.20 |
| 2′ | 4.84 | 4.88, 4.93 | 4.86 | 4.94 |
| 3′ | 4.51 | 4.64, 4.67 | 4.49 | 4.72 |
| 4′ | 4.35 | 4.57-4.62, 4.60-4.65 | 4.38 | 4.58-4.67 |
| 5a′ | 3.05 | 3.54, 3.61 | 3.05 | 3.84-3.95 |
| 5b′ | 3.00 | 3.54, 3.55 | 3.03 | 3.65 |
| Ar | 8.43 | 8.42 | 8.44 | 8.45 |
| Ar | 8.48 | 8.43, 8.46 | 8.50 | 8.48 |

Characterization of selenoxide SeAHO in an aqueous environment was of greater biological significance. The selenoxide SeAHO was completely stable to hydrolysis of the Cl′-adenine bond in phosphate buffered aqueous solutions for at least three hours at ambient temperature over the wide pH range of 3-12 as no elimination or other degradation products were seen by HPLC or proton NMR. The SeAHO prepared in phosphate buffers at pHs 3, 7 and 12 each gave homogeneous chromatograms by reversed-phase HPLC (data not shown), although some decomposition was observed by HPLC after 2 weeks or when prepared in pure deionized water.

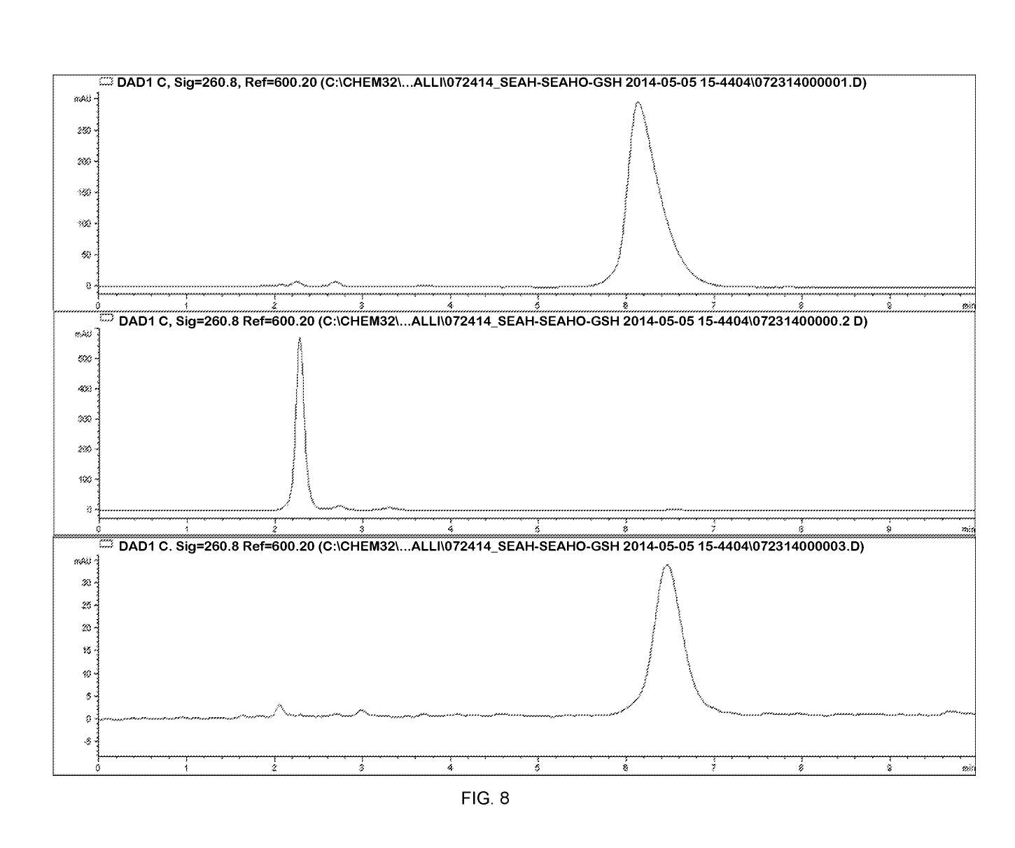

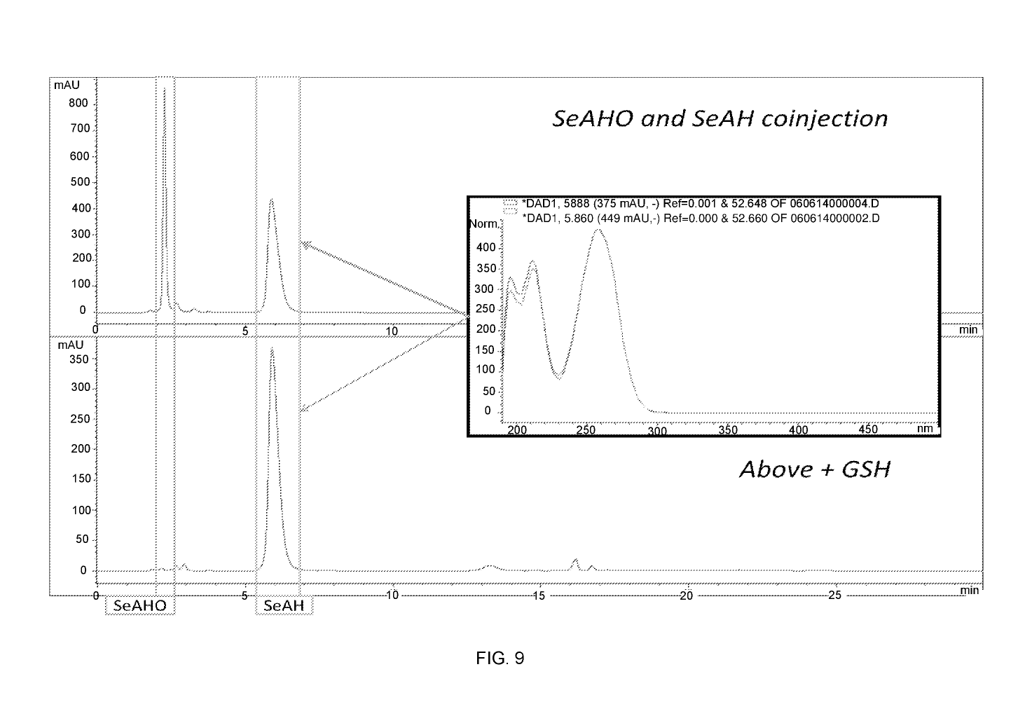

The selenoxide SeAHO was readily reduced back to selenide SeAH at ambient temperature by glutathione (GSH) as observed by C18 HPLC (150×4.6 mm, 260 nm, 0.1% formic acid, 2:98 acetonitrile/water, 1 mLmin⁻¹). See FIGS. 8-9. The selenoxide SeAHO was also reduced by cysteine, but not by thioethers methionine or SAH (data not shown). The sulfoxide SAHO analog was not reduced under these biological conditions with glutathione (GSH) or cysteine. The reduction of sulfoxides generally requires more forcing conditions.

The selenoxide SeAHO generally appeared to be a 60:40 mixture by ¹H NMR in 50 mM phosphate buffers of D₂O at measured pH's of 3 and 7. High resolution NMR at pH 7 showed that 40% of the material had the α-proton shifted downfield, and that the selenoxide SeAHO was also a 50:50 mixture, likely to be a mix of selenoxide and hydrate, analogous to the reported data for selenomethionine selenoxide (Zainal, H. A.; Wolf, W. R.; Waters, R. M. J. Chem. Technol. Biotechnol. 1998, 72, 38-44; Block, E.; Birringer, M.; Jiang, W.; Nakahodo, T.; Thompson, H. J.; Toscano, P. J.; Uzar, H.; Zhang, X.; Zhu, Z. J. Agric. Food Chem. 2001, 49, 458-70; Ritchey, J. A.; Davis, B. M.; Pleban, P. A.; Bayse, C. A. Org. Biomol. Chem. 2005, 3, 4337-42). The selenoxide SeAHO was presumably mostly hydrate at pH 3 and mostly in the selenoxide form at pH12 where only a very small amount of decomposition was observed.

The coordination of the α-amino acid moieties with the selenoxide functional group of the Se-methyl analog, selenomethionine selenoxide, in aqueous solutions have already been proposed at acidic, neutral, and basic pHs. The protonation, hydration, and racemization of the selenoxide functional group at low pH is also well-known. These data correlate well with our NMR data for Se-adenosylselenohomocysteine selenoxide (SeAHO) in the acidic organic (acetic acid-d₄) and in the phosphate buffered aqueous environments at pHs 3, 7, and 12. The characteristic coordination of the selenoxide (SeAHO) can be intermolecular or intramolecular. The interaction of the α-amino acid moiety of SeAHO with the selenoxide/hydrate group results in deshielding of the α-proton completely in acetic acid-d₄ and to the extent of about 40% in acidic and neutral buffered aqueous solutions.

The mass spectra of selenoxide SeAHO were obtained by LC-MS (ToF) and LCQ-MS (ion trap) in an acidic environment (acetonitrile/water/0.1% formic acid), as well as by MALDI (matrix of α-cyano-4-hydroxycinnamic acid and trifluoroacetic acid). The molecular ion of the selenoxide SeAHO m/z 449 (M+H)⁺ was observed by the LCQ-MS and MALDI techniques, and hydrated [—Se⁺(OH)—][⁻OH] and/or dihydroxyseleno —Se(OH)₂— ion with an m/z 467 (M+H₂O+H)⁺ was also seen by the softer MALDI ionization technique. The MS-MS fragmentations of the m/z 449 and 467 ions were distinct from the fragmentation of the base peak m/z 431 seen by the LC-MS, LCQ-MS, and MALDI techniques. Under the various ionization conditions, cyclic analogs and/or eliminations to give [—Se⁺=] species can account for the m/z 431. MALDI MS-MS of the m/z 431 ion gives the m/z 250 ion (5′-adenosyl cation) resulting from cleavage of the selenium-C5′ bond and also m/z 136 (adenine+H)⁺ ion.

Conclusions: Se-Adenosylselenohomocysteine selenoxide (SeAHO) was synthesized from adenosine by a method that did not require any extractions or column chromatography. Selenoxide SeAHO was stable in buffered aqueous environments with no evidence of glycosidic hydrolysis or electrocyclic eliminations over a wide (3-12) pH range at ambient temperature. This selenoxide (SeAHO) has not yet been characterized from biological samples, perhaps due to low abundance in cellular reducing environments and a weak molecular ion in the MS. Selenoxide SeAHO is quite distinct from its sulfoxide (SAHO) analog. Selenoxide SeAHO is readily reduced by biological reductants glutathione (GSH) and cysteine thiols, it undergoes hydration at the larger more polarizable selenium, and is racemized at the selenium center at low pH. The greater conformational flexibility of the selenoxide analog (SeAHO) was seen in the proton NMR chemical shift of the α-proton, likely due to electrostatic interactions of the amino acid moieties with the selenoxide functional group. Due to the close structural similarity to the sulfoxide (SAHO) analog, the selenoxide SeAHO should also be an inhibitor and/or activator for S-adenosylmethionine-dependent methyltransferases, other enzymes, and proteins.

### Example 2

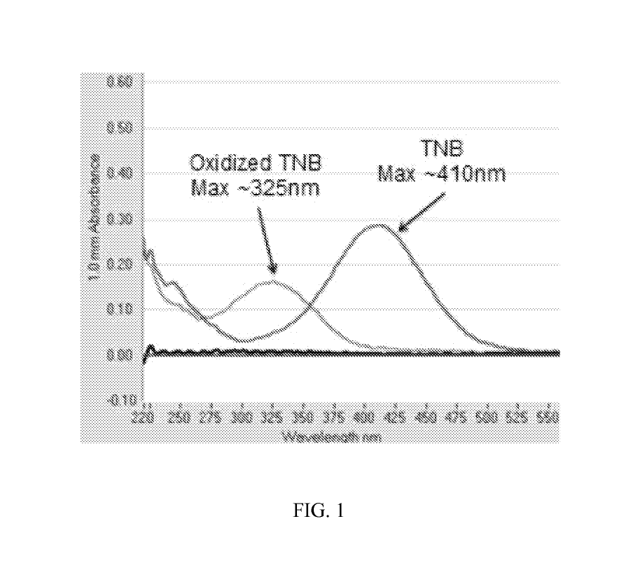

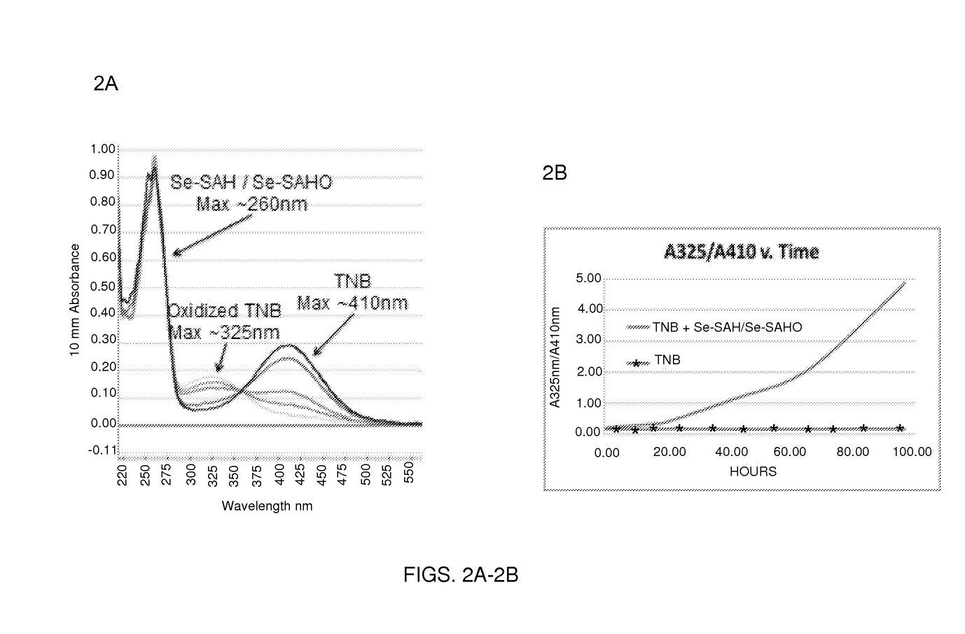

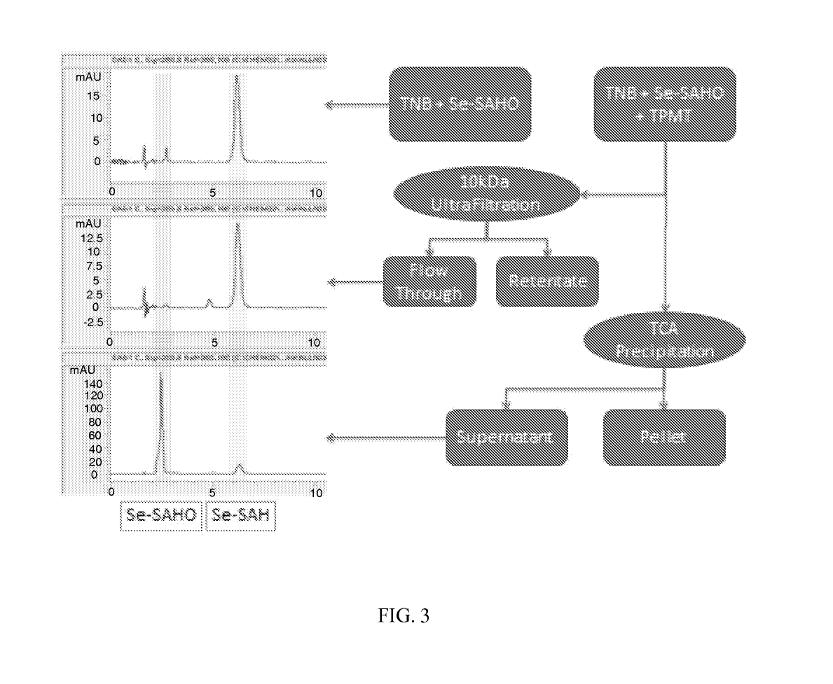

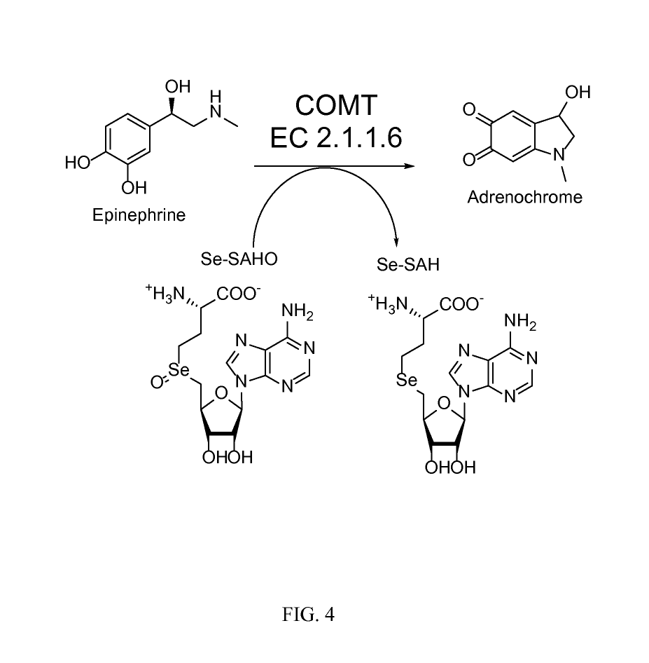

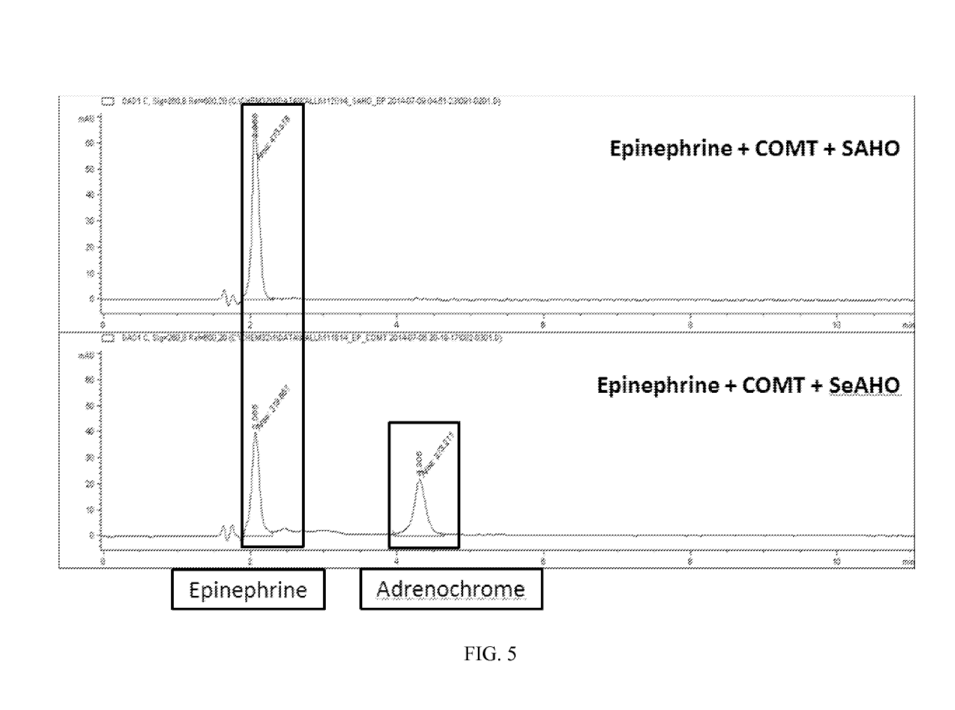

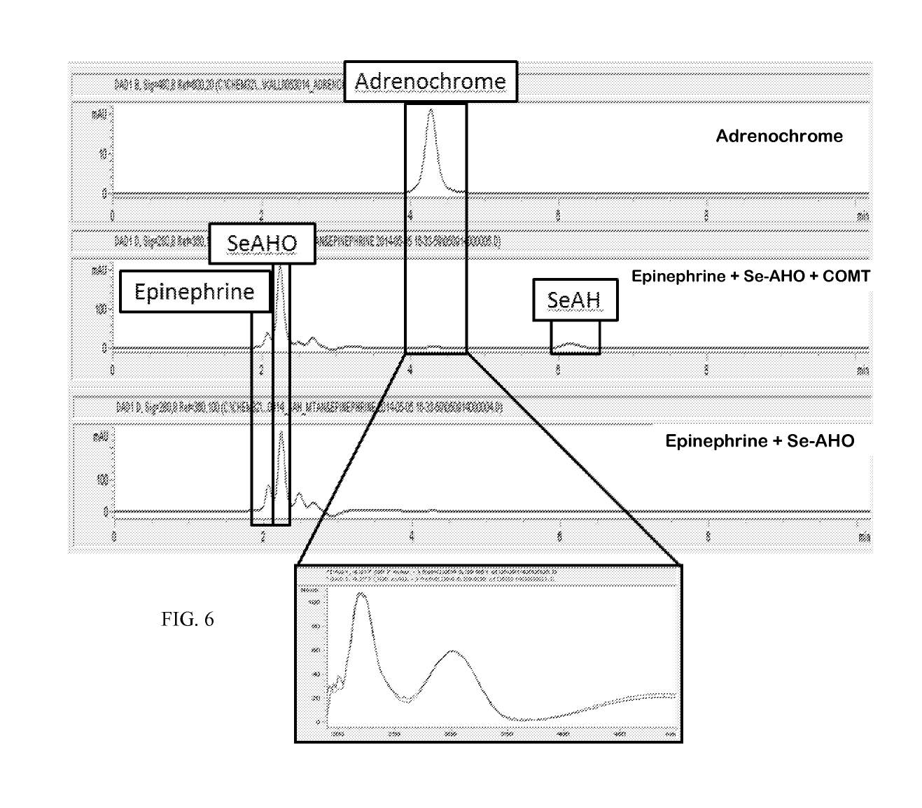

SeAHO has been shown to be reduced to Se-adenosyl-L-selenohomocysteine (SeAH) upon incubation with certain methyltransferases, e.g. catechol-O-methyltransferase (COMT) and thiopurine methyltransferase (TPMT). See FIGS. 1-6. However, SeAHO was not reduced upon incubation with the non-methyltransferase protein lysozyme (data not shown). This activity was not observed in the corresponding sulfoxide compound (SAHO), which remains oxidized after exposure to COMT (data not shown). The reduction of SeAHO by methyltransferases may result in the oxidation or modification of these enzymes. This property could be exploited to identify methyltransferases that are reactive with SeAHO, or, depending on the site of oxidation, this process may modulate the activities of methyltransferases. In other words, SeAHO may act as a covalent inhibitor (reactive or suicide inhibitor) or activator of these methyltransferases.

### Example 3

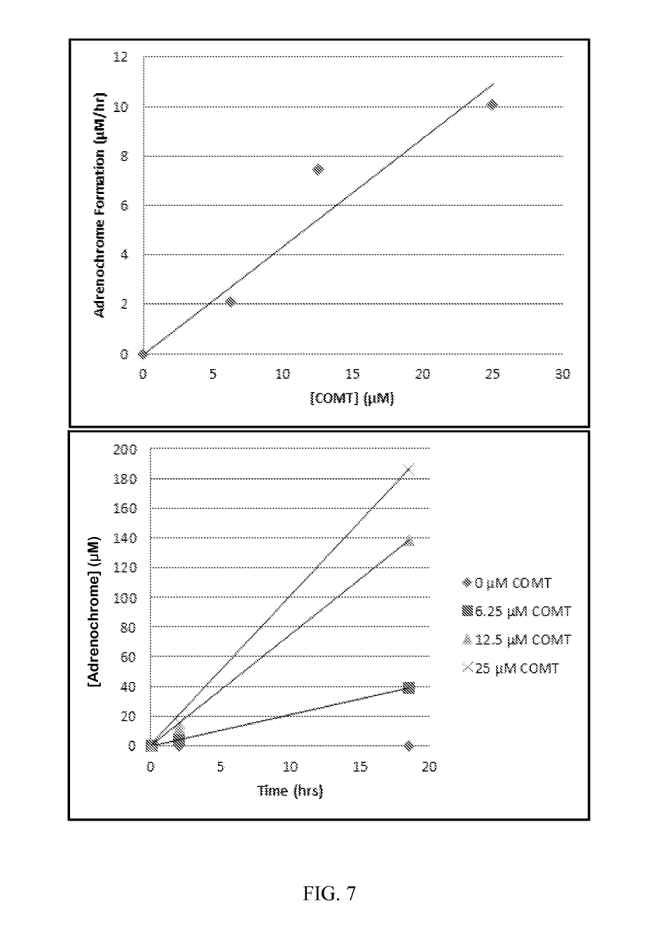

Catechol-O-methyltransferase (COMT), which normally methylates catechol-containing substrates, catalyzes the oxidation of epinephrine with SeAHO and the production of adrenochrome and SeAH. See FIG. 7. This reactivity (redox reaction) conferred by SeAHO can be exploited to oxidize substrate compounds in a selective and specific manner.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A compound having a structural formula: 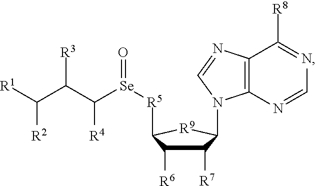
or a hydrate or an isotope thereof, wherein
R¹ is COOH, NH₂, CH₂OH, CHO, CONH₂, Cl, Br, I, H, O, OH, N₃, CH₃ or CN;
R² is NH₂, COOH, CH₂OH, CHO, CONH₂, Cl, Br, I, H, O, OH, N₃, CH₃ or CN;
R³ is H or a linear or branched C₁-C₄ alkyl;
R⁴ is H or a linear or branched C₁-C₄ alkyl;
R⁵ is CH₂, —CH(CH₃), C(CH₃)₂ or C₂H₄;
R⁶ is OH, O or a linear or branched C₁-C₄ alkoxy;
R⁷ is OH, O or a linear or branched C₁-C₄ alkoxy;
R⁸ is NH₂, COOH, Cl, Br, I, H, O, OH, N₃, CH₃ or CN; and
R⁹ is O, N, S or CH₂.

2. The compound of claim 1, wherein the compound has a structural formula:

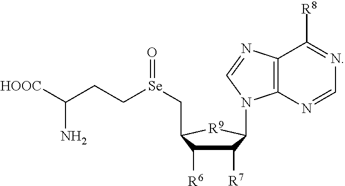

3. The compound of claim 1, wherein the compound has a structural formula:

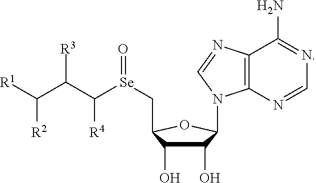

4. The compound of claim 1, wherein the compound has a structural formula:

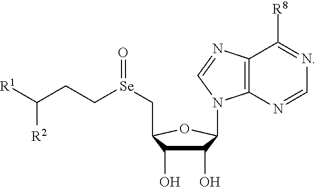

5. The compound of claim 1, wherein the compound has a structural formula:

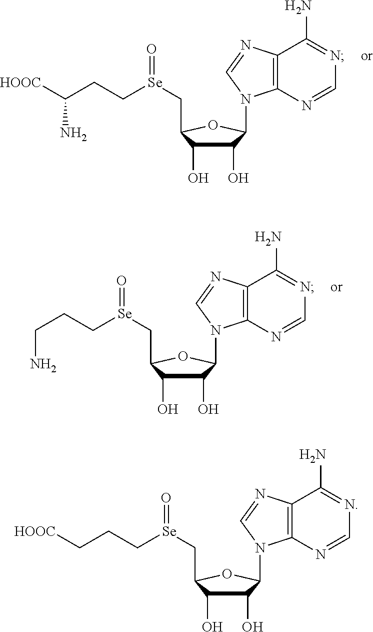

6. The compound of claim 1, wherein the hydrate of the compound has a structural formula:

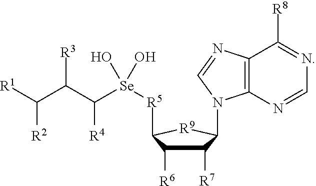

7. The compound of claim 1, wherein the compound is represented by one of:

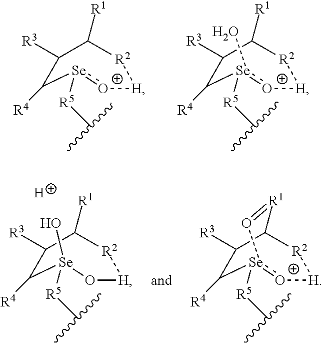

8. A method of preparing a compound of claim 1, the method comprising oxidizing a precursor compound having a structural formula: 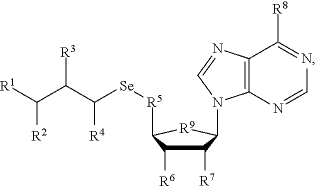
thereby obtaining the compound of claim 1.

9. The method of claim 8, wherein R¹ is COOH, R² is NH₂, R³ is H, R⁴ is H, R⁵ is CH₂, R⁶ is OH, R⁷ is OH, R⁸ is NH₂ and R⁹ is O.

10. The method of claim 9, wherein the method further comprises, before the oxidizing:

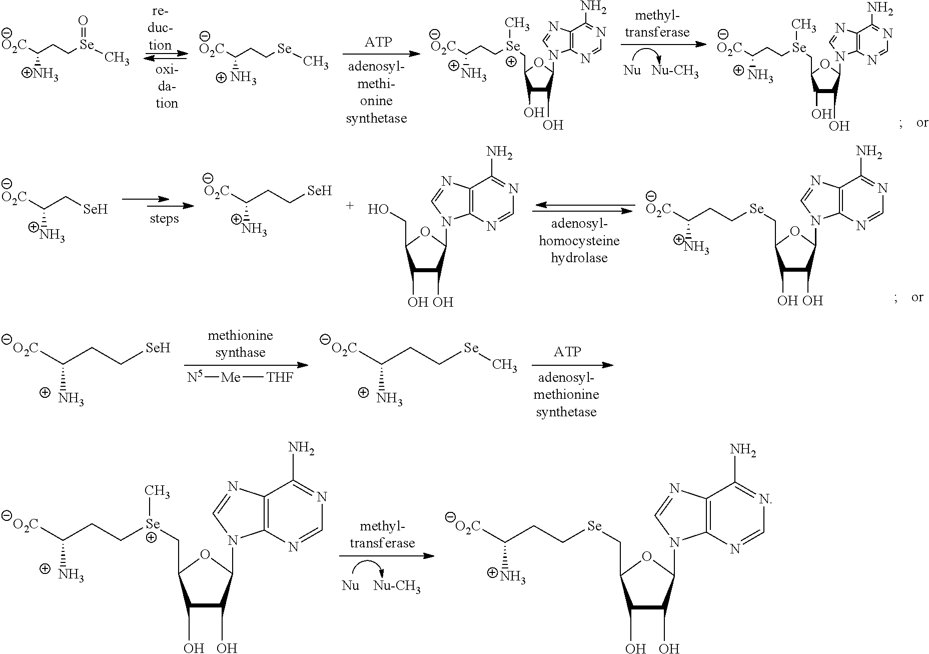

11. The method of claim 9, wherein the method further comprises, before the oxidizing:

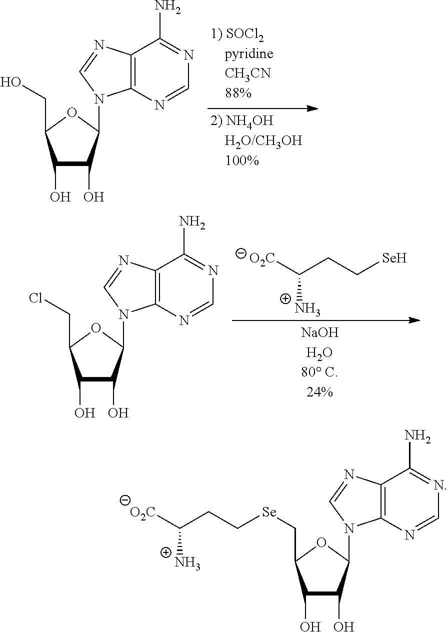

12. The method of claim 11, wherein the method is performed without extraction and without column chromatography.

13. A method of providing dietary organoselenium to a subject in need thereof, the method comprising administering to the subject a composition comprising the compound of claim 1.

14. The method of claim 13, wherein the subject has a selenium imbalance.

15. A method of inactivating an enzyme, the method comprising contacting, under physiological conditions, the enzyme with the compound of claim 1, wherein the enzyme has at least one of an accessible cysteine or oxidizable functional group in an active site of the enzyme.

16. The method of claim 15, wherein the enzyme having an accessible cysteine group in an active site of the enzyme is adenosyl homocysteine hydrolase.

17. The method of claim 15, wherein the enzyme having an oxidizable functional group in an active site of the enzyme is DNA(cytosine-5)-methyltransferase 1.

18. The method of claim 15, wherein the enzyme is an enzyme that binds S-adenosylmethionine.

19. The method of claim 18, wherein the enzyme is a methyltransferase.

20. A method of modulating the activity of a methyltransferase, the method comprising contacting, under physiological conditions, the methyltransferase with the compound of claim 1.

21. The method of claim 20, wherein the methyltransferase is a S-adenosylmethionine dependent methyltransferase.

22. The method of claim 20, wherein the methyltransferase is at least one of catechol-0- methyltransferase and thiopurine methyltransferase.

23. The method of claim 20, wherein the compound inhibits the activity of a methyltransferase.

24. The method of claim 20, wherein the compound activates the activity of a methyltransferase.

25. A method of identifying a methyltransferase that reacts with a compound of claim 1, the method comprising:
a) contacting, under physiological conditions, a methyltransferase with the compound of claim 1, thereby oxidizing the methyltransferase and producing a 16 Da mass shift in the methyltransferase, and
b) identifying the methyltransferase by the 16 Da mass shift.

26. A method of oxidizing a methyltransferase-reactive substrate, the method comprising contacting, under physiological conditions, the substrate with a methyltransferase and the compound of claim 1.
